(19) United States
(12) Patent Application Publication
(10) Pub. No.: US 2019/0022424 A1
(43) Pub. Date: Jan. 24, 2019

(54) FOCUSED ULTRASOUND STIMULATION APPARATUS USING USER CUSTOMIZED ACOUSTIC LENS

(71) Applicant: KOREA INSTITUTE OF SCIENCE AND TECHNOLOGY, Seoul (KR)

(72) Inventors: Hyung Min KIM, Seoul (KR); Ki Joo PAHK, Seoul (KR); Inchan YOUN, Seoul (KR); Seung-Jong KIM, Seoul (KR); Yoha HWANG, Seoul (KR)

(73) Assignee: KOREA INSTITUTE OF SCIENCE AND TECHNOLOGY, Seoul (KR)

(21) Appl. No.: 15/868,629

(22) Filed: Jan. 11, 2018

(30) Foreign Application Priority Data
Jul. 20, 2017 (KR) 10-2017-0092005

(51) Int. Cl. A61N 7/00

(52) U.S. Cl. CPC A61N 7/00; A61N 2007/0086; A61N 2007/006; A61N 2007/0026

(57) ABSTRACT

A focused ultrasound stimulation apparatus according to the present disclosure includes a transducer which outputs low intensity/high intensity ultrasound, an acoustic lens which is placed in close contact with a user's skin and is customized to focus the ultrasound onto a target focal point, and a fixture for fixing the transducer and the acoustic lens to each other. The acoustic lens is customized using a 3-dimensional (3D) printer based on the pre-captured user's cranial shape, to focus the ultrasound a desired focus target for each user, thereby improving accuracy compared to conventional ultrasound stimulation apparatus.

### CROSS-REFERENCE TO RELATED APPLICATION

This application claims priority to Korean Patent Application No. 10-2017-0092005, filed on Jul. 20, 2017, and all the benefits accruing therefrom under 35 U.S.C. § 119, the contents of which in its entirety are herein incorporated by reference.

### BACKGROUND

### 1. Field

The present disclosure relates to a focused ultrasound stimulation apparatus, and more particularly, to a focused ultrasound stimulation apparatus for treating a disease by focusing ultrasound onto a target focal point using an acoustic lens which is customized based on the pre-captured user's cranial shape.

### DESCRIPTION OF GOVERNMENT-SPONSORED RESEARCH AND DEVELOPMENT

This research is done in support of study-centered hospital advancement program (Development of non-invasive ultrasound based neuromodulation and muscle rehabilitation system, Project series number: 1460523070) of Ministry of Health and Welfare under the supervision of Korea Institute of Science and Technology.

### 2. Description of the Related Art

To perform a treatment method for relieving a patient's pain or stimulating a neural cell of a particular body part, conventionally, a method which inserts electrodes into the patient's body was used, but this physical invasive operation has a risk that the body may be damaged.

Recently, ultrasound stimulation treatment for stimulating a wound part without a physical invasive operation is being widely used. Ultrasound can be classified into high intensity ultrasound and low intensity ultrasound according to the intensity, and the high intensity ultrasound is used for direct treatment such as cancer cell or tissue necrosis, whereas the low intensity ultrasound is known as gaining a medical effect without applying heat to the body or causing tissue necrosis.

The unit of ultrasound intensity is indicated in spatial-peak temporal-average intensity (Ispta) and spatial-peak pulse average intensity (Isppa) based on Acoustic Output Measurement Standard for Diagnostic Ultrasound Equipment by American Institute for Ultrasound in Medicine and National Electronics Manufacturers Administration (NEMA).

Although there is no clearly defined standards about the type of ultrasound yet, “low intensity ultrasound” as used herein refers to ultrasound with Ispta of less than 3 W/cm² in accordance with U.S. FDA standards and European safety standards and which is within the range that does not cause damage to the body, and ultrasound with Ispta of 3 W/cm² or above is defined as “high intensity ultrasound”.

Of them, a low intensity ultrasound stimulation apparatus is being widely used to stimulate a patient's brain neural cell in order to treat mental diseases such as a depressive disorder and schizophrenia, or epilepsy and hand tremor. However, the conventional ultrasound stimulation apparatus for brain stimulation has a problem that ultrasound generated from a transducer reduces in focality, or changes in a path along which ultrasound travels due to a medium difference (i.e., acoustic impedance mismatch) while passing through the patient's skull and brain, failing to focus ultrasound onto a target focal point, and to solve the problem, it was general to use multiple transducer arrays.

Referring to Korean Patent No. 10-1700883, there is disclosed an apparatus for stimulating a patient's spinal cord or neural cell around the spinal cord using a focused ultrasound array including multiple transducers to output a low intensity ultrasound beam. Furthermore, an example of the use of ultrasound for brain stimulation is INSIGHTEC Ltd.'s transcranial ultrasound treatment device ExAblate Neuro, composed of tens of transducers, whereby it has a focusing advantage, while high price is a disadvantage.

To solve the high price problem of the multichannel ultrasound stimulation apparatus, a method was designed to focus ultrasound generated from a single channel ultrasound apparatus by use of an acoustic lens. Referring to WO2013/166019 A1, a focused ultrasound apparatus is disclosed in which an acoustic lens designed using a 3-dimensional (3D) printer is attached in front of a transducer, to improve the focality of ultrasound.

However, in the related art, much process costs are required to manufacture such that the acoustic lens is attached in front of the transducer, and the acoustic lens is manufactured attachably to the transducer, not fit a patient's body part, so it only has an effect in simply focusing ultrasound.

### SUMMARY

Therefore, the disclosure is directed to providing an ultrasound stimulation apparatus for focusing ultrasound onto a desired focus target using an acoustic lens which is customized based on the user's cranial shape pre-captured by a computed tomography (CT) or magnetic resonance imaging (MRI) imaging apparatus.

Furthermore, the disclosure is directed to providing an ultrasound stimulation apparatus with reduced cost by easily replacing an acoustic lens suitable for each target patient, and with reduced cost by replacing an acoustic lens or adding a second acoustic lens with the change in focus target.

A focused ultrasound stimulation apparatus according to an embodiment includes a transducer which outputs ultrasound, an acoustic lens which is placed in close contact with a user's skin and is customized to focus the ultrasound onto a target focal point, and a fixture for fixing the transducer and the acoustic lens to each other.

In an embodiment, the acoustic lens may be customized using a 3-dimensional (3D) printer based on a pre-captured user's cranial shape.

In an embodiment, the acoustic lens may be customized to focus the ultrasound separately onto at least two target focal points.

In an embodiment, the fixture may be configured to attach and detach the acoustic lens.

In an embodiment, the fixture may be configured to insert a second acoustic lens between the transducer and the acoustic lens, wherein the second acoustic lens is customized to focus the ultrasound separately onto a target focal point different from the target focal point.

In an embodiment, the focused ultrasound stimulation apparatus may further include a third acoustic lens which is attached in front of the transducer to improve focality of the ultrasound.

In an embodiment, the fixture may be filled with a medium made of hydrogel or water.

In an embodiment, the acoustic lens may be made of at least one of polymers, elastomers and polydimethylsiloxane (PDMS).

According to the focused ultrasound stimulation apparatus according to an embodiment as provided herein, accuracy can be improved compared to the conventional ultrasound stimulation apparatus by focusing low intensity/high intensity ultrasound onto a desired focus target using the acoustic lens which is customized based on the user's cranial shape pre-captured by a CT or MRI imaging apparatus.

Furthermore, it is possible to stimulate multiple target focal points with a single transducer using acoustic lenses with different structures, so the same effect as the use of multiple transducers or multi-array transducer can be obtained.

Additionally, because the fixture of the ultrasound stimulation apparatus has a structure that is easy to detach and attach or add the acoustic lens, any possible extra costs incurred with the change in target patient or focus target can be reduced.

### BRIEF DESCRIPTION OF THE DRAWINGS

FIG. 1 schematically shows the structure of a focused ultrasound stimulation apparatus according to an embodiment.

FIG. 2 is a diagram illustrating the configuration of a transducer according to an embodiment.

FIGS. 3A-3C are diagrams illustrating the piezoelectric effect of a piezoelectric element.

FIG. 4A is a diagram showing the focusing of ultrasound onto a target focal point when a conventional ultrasound stimulation apparatus is used in ideal situation.

FIG. 4B is a diagram showing the failed focusing of ultrasound onto a target focal point when a conventional ultrasound stimulation apparatus is used in real situation.

FIG. 5 is a diagram showing the accurate focusing of ultrasound onto a target focal point when a focused ultrasound stimulation apparatus according to an embodiment is used.

FIG. 6 is a diagram showing the focusing of ultrasound onto multiple target focal points when a focused ultrasound stimulation apparatus according to an embodiment is used.

FIG. 7 is a diagram showing a second acoustic lens inserted into a fixture in a focused ultrasound stimulation apparatus according to an embodiment.

### DETAILED DESCRIPTION

The present disclosure is described in detail as below with reference to the accompanying drawings in which particular embodiments for carrying out the present disclosure are shown for illustration. These embodiments are described in sufficient detail to enable those skilled in the art to practice the present disclosure. It should be understood that various embodiments of the present disclosure are different from each other, but they do not need to be exclusive. For example, a particular shape, structure and characteristic described herein, in connection with one embodiment, may be implemented in other embodiments without departing from the spirit and scope of the present disclosure. It should be further understood that modification may be made to the position or arrangement of respective elements in each disclosed embodiment without departing from the spirit and scope of the present disclosure. Therefore, the following detailed description is not intended to make in a limitative sense, and the scope of the present disclosure is only defined by the appended claims, if appropriately described, along with the full scope of equivalents to which the claims are entitled. In the drawings, similar reference numerals denote same or similar functions throughout many aspects.

Hereinafter, the preferred embodiments of a focused ultrasound stimulation apparatus will be described in more detail with reference to the accompanying drawings.

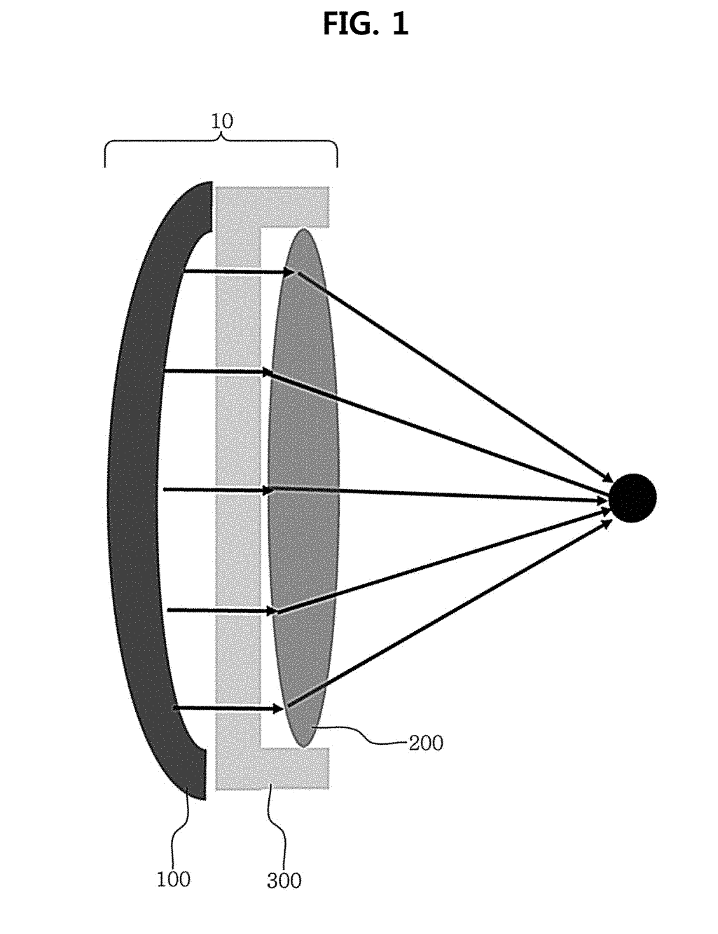

FIG. 1 is a diagram schematically showing the structure of the focused ultrasound stimulation apparatus according to an embodiment. In the embodiment, the focused ultrasound stimulation apparatus 10 includes a transducer 100, an acoustic lens 200 and a fixture 300.

The transducer 100 is a sound source which outputs ultrasound. In the embodiment, the transducer 100 can output 3 W/cm² (Ispta) or less of low intensity ultrasound as well as 3 W/cm² (Ispta) or above of high intensity ultrasound by adjusting the output based on the regions to treat and the purpose.

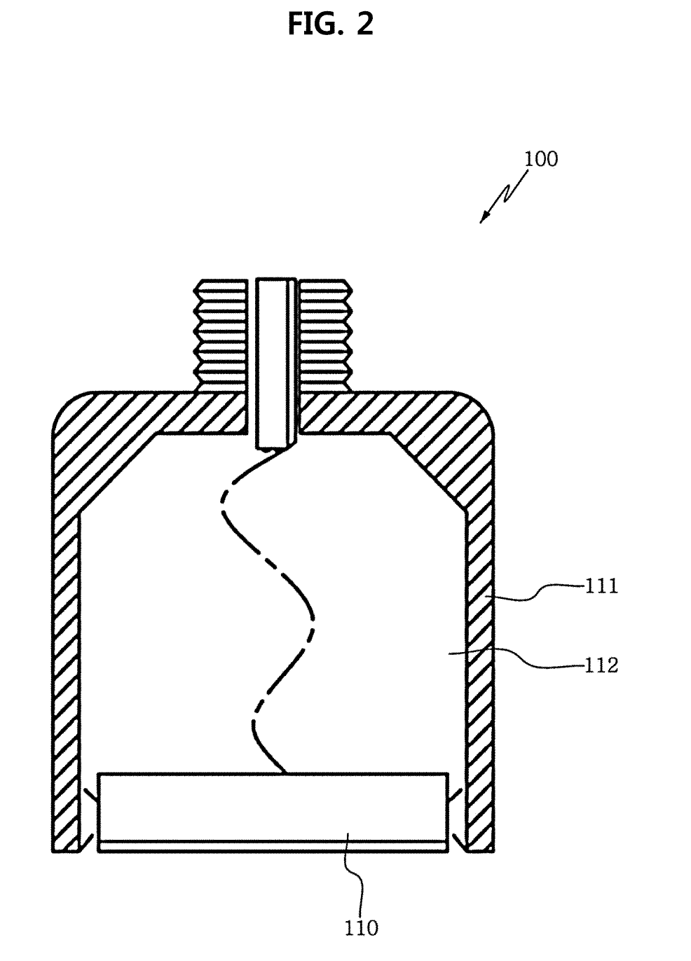

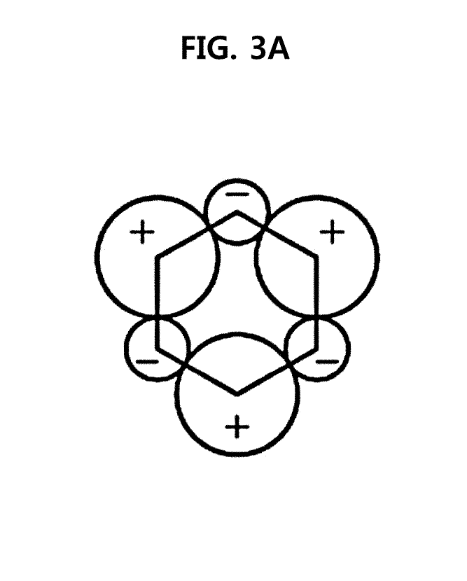

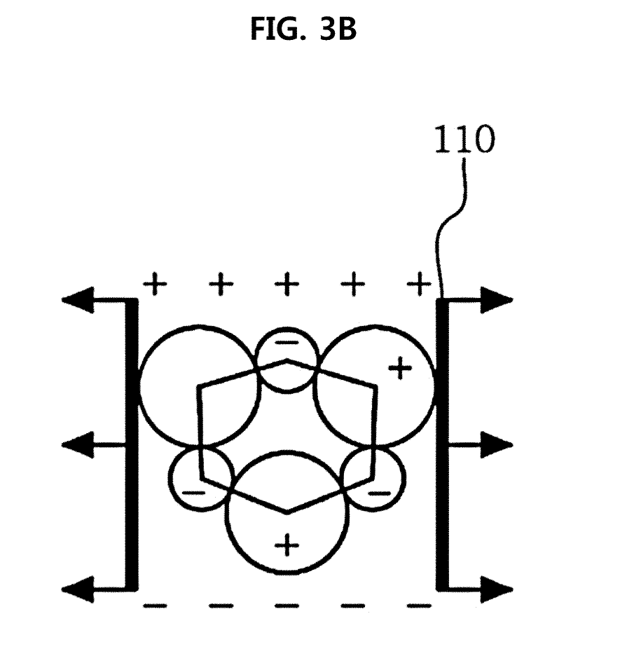

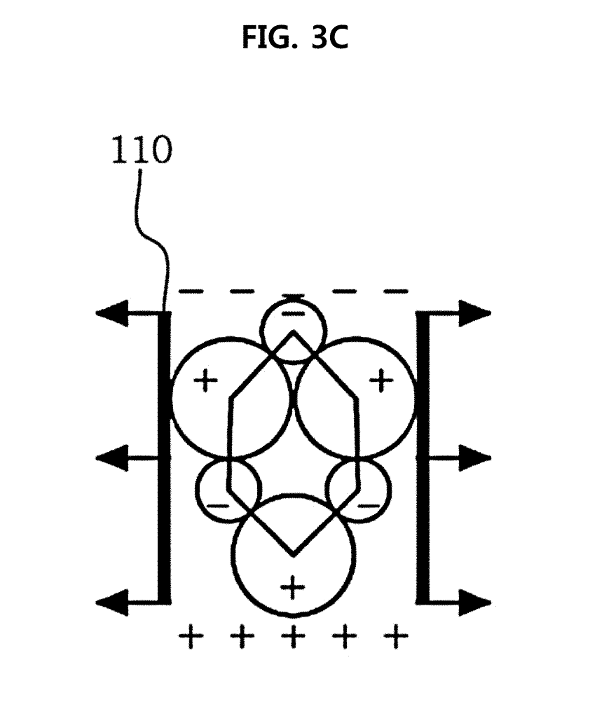

In general, an ultrasonic transducer converts 20 kHz or above of alternating energy to mechanical vibration of the same frequency by the application of the piezoelectric effect or magnetostrictive effect. The detailed structure of the transducer 100 and the ultrasound generation mechanism using the piezoelectric element will be described in detail as below with reference to FIGS. 2 and 3A-3C.

FIG. 2 is a diagram illustrating the configuration of the transducer 100 according to an embodiment. However, the structure of the transducer is for illustration only, and is not limited to a particular structure or effect.

As shown in FIG. 2, the transducer 100 according to this embodiment includes a body 111 that is open to one side and a piezoelectric element 110 formed at the opening of the body 111. An inside of the body 111 is filled with air 112. An electric wire is connected to each piezoelectric element 110 to apply voltage to the piezoelectric element 110. The body 111 may be elongated to simultaneously fix multiple piezoelectric elements 110, and may be sized to fix one piezoelectric element 110.

According to this embodiment, the piezoelectric element 110 exploits a material that exhibits the piezoelectric effect such as quartz and turmaline, and the transducer 100 produces and outputs ultrasound using the piezoelectric effect of the piezoelectric element 110.

FIGS. 3A-3C are diagrams illustrating the piezoelectric effect of the piezoelectric element 110. As shown, when tension and compression is repeatedly applied along one axis of the piezoelectric element 110 made of quartz crystals, positive charge (+) is created on one side and negative charge (−) is created on the other side, producing an electric current.

This polarization phenomenon of the piezoelectric element 110 occurs due to a change in relative position of (+) ions and (−) ions as the crystal structure distorts. Thus, the center of gravity of charges having moved the position in the element is automatically corrected, but an electric field is formed across the crystal. The electric field is in opposite directions under compression and tension.

On the contrary, when voltage is applied across the piezoelectric element 110, (+) ions in the electric field move to (−) electrode, and (−) ions move to (+) electrode. By the inverse piezoelectric effect, tension and compression is induced in the piezoelectric element 110 based on the direction of voltage applied from the outside. As the piezoelectric element 110 is repeatedly subjected to tension and compression, ultrasound with frequencies above the audible range is produced in the similar principle to the operation principle of a speaker.

The piezoelectric element 110 of the transducer 100 can output a proper intensity of ultrasound by adjusting the output based on the regions to treat and the purpose, and the outputted ultrasound causes an overlap to form an ultrasound beam.

In an embodiment, a function module including a signal generator that generates a voltage signal of the transducer 100 being applied and an amplifier that amplifies the signal may be additionally connected to the ultrasound stimulation apparatus 10. Furthermore, a computer that can regulate the function module and output various signals to a monitor may be further connected.

The acoustic lens 200 is customized to be placed in close contact with a user's skin (for example, forehead), so as to focus the ultrasound outputted by the transducer 100 onto a target focal point (for example, point in a skull). The ultrasound reduces in output and changes in path while passing through different media such as the patient's skull or brain, and the acoustic lens 200 serves as an assistant to compensate for the reduced focality and focus the ultrasound onto a correct target focal point.

To this end, the user's cranial shape is pre-captured through computed tomography (CT) imaging and/or magnetic resonance imaging (MRI) imaging, and the acoustic lens configured to accurately focus ultrasound onto a target focal point based on the captured cranial shape is manufactured. In the embodiment, the acoustic lens may be made of one of polymers, elastomers and polydimethylsiloxane (PDMS), but is not limited thereto.

According to embodiments, the acoustic lens may be manufactured using 3-dimensional (3D) printing technique. The 3D printer used in manufacturing the acoustic lens includes all devices that make stereoscopic products based on 3D diagram by up-down (z axis) motions in addition to back-forth (x axis) motions and left-right (y axis) motions, and both a layering-type 3D printer that stacks one layer on another and a cutting-type 3D printer that cuts away from a block of material may be used.

In case that the acoustic lens is manufactured using the 3D printer, the production cost is low, and if patient information captured using CT or MRI is provided, a custom shaped lens that can be placed in close contact with the patient's head is manufactured, thereby maximizing the ultrasound focusing effect of the acoustic lens.

The fixture 300 is a component for fixing the transducer 100 and the acoustic lens 200 to each other. The fixture 300 may be formed with a hollow cylindrical structure not to affect the path of ultrasound generated from the transducer 100, but is not limited to a particular shape. Furthermore, in the embodiment, the inside of the fixture may be filled with a medium such as hydrogel or water according to necessity, but is not limited thereto.

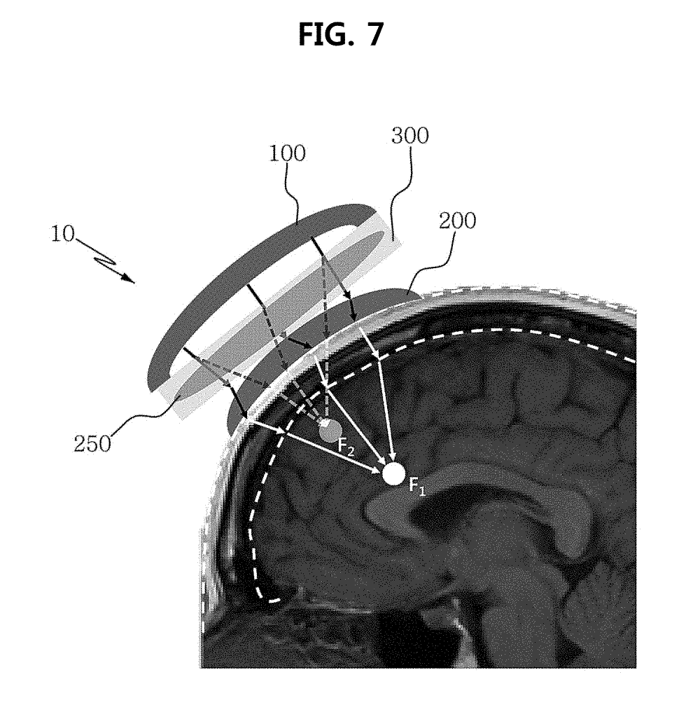

According to embodiments, the fixture 300 may be configured to attach and detach the acoustic lens 200, or may have a structure allowing a second acoustic lens (250 in FIG. 7) to be inserted between the transducer 100 and the acoustic lens 200. A description of additional functions and shapes of the fixture will be provided with reference to the drawings later.

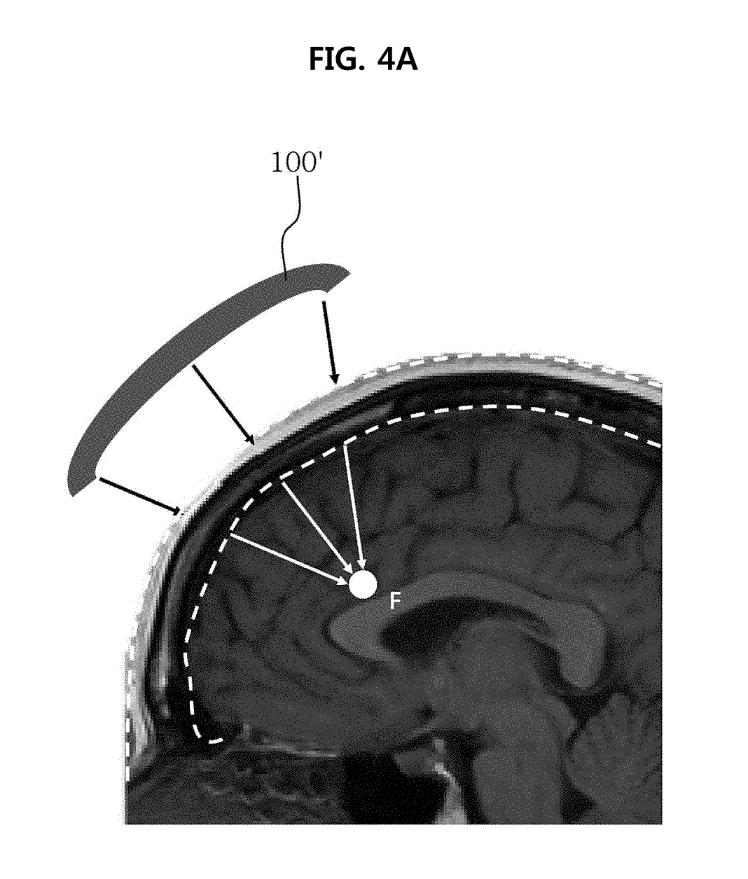

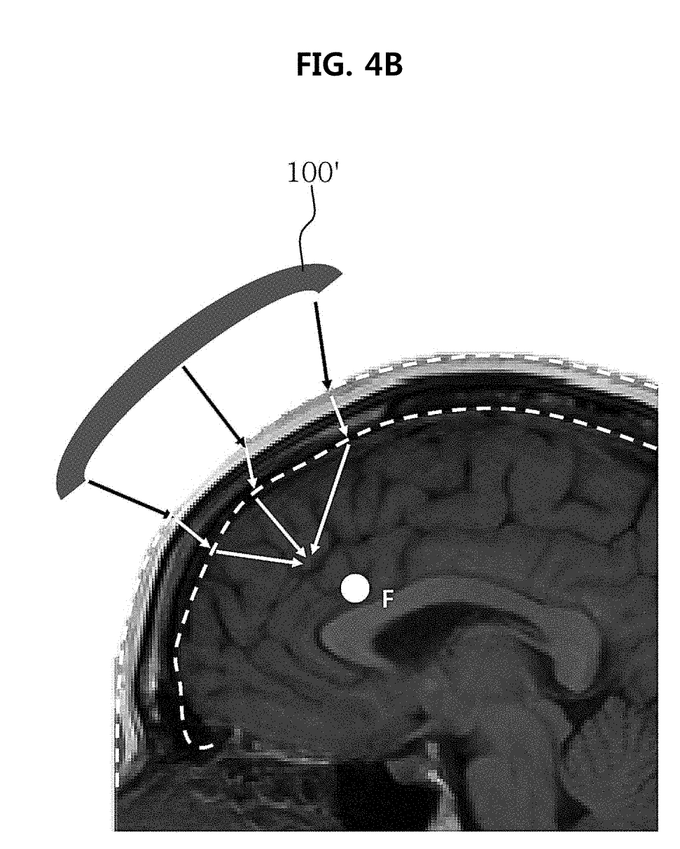

Subsequently, referring to FIGS. 4A and 4B, the conventional ultrasound stimulation apparatus with no use of an acoustic lens and the ultrasound stimulation apparatus using the customized acoustic lens according to this embodiment will be compared.

FIG. 4A is a diagram showing the case of using the conventional ultrasound stimulation apparatus in ideal situation, and FIG. 4B is a diagram showing the failed focusing of ultrasound onto a target focal point F when the conventional ultrasound stimulation apparatus is used in real situation.

Ideally, ultrasound generated from the transducer 100′ is expected to be focused onto the target focal point F through the skull as shown in FIG. 4A, but in practice, ultrasound changes in path due to a medium difference while passing through the patient's skull and brain, and the focusing onto the target focal point F fails as shown in FIG. 4B. Furthermore, in the case of the conventional ultrasound stimulation apparatus with no acoustic lens, fine adjustment is impossible, resulting in low focality of ultrasound.

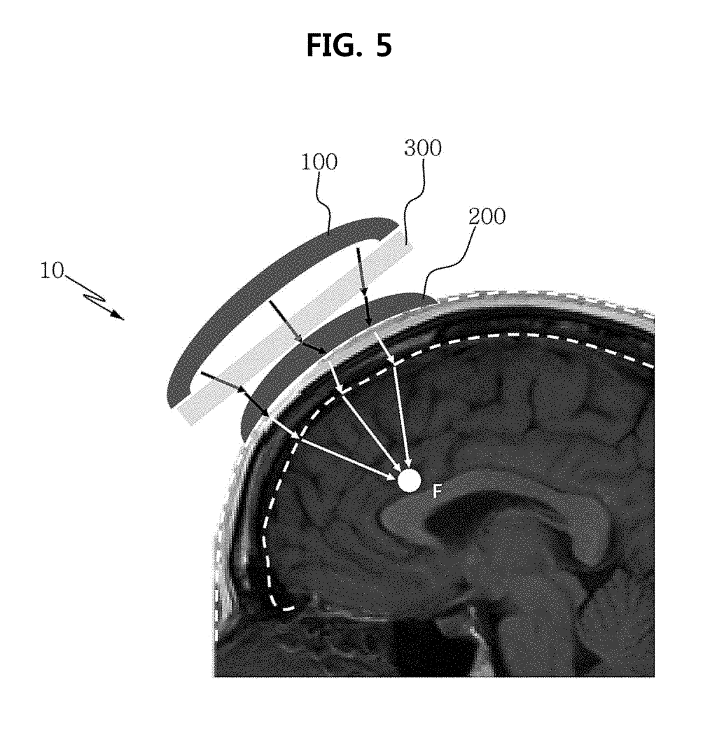

In the case of using the ultrasound stimulation apparatus according to the embodiment provided herein, as shown in FIG. 5, ultrasound outputted from the transducer 100 can be accurately focused onto the target focal point F along the path corrected by the acoustic lens 200 placed in close contact with the head, and focality can also be improved. As described above, the acoustic lens 200 may be formed with a structure allowing the focusing of ultrasound onto the target focal point F, taking into account the pre-captured shape of the user's skull and brain, and may be manufactured using 3D printing technique based on the pre-acquired image information.

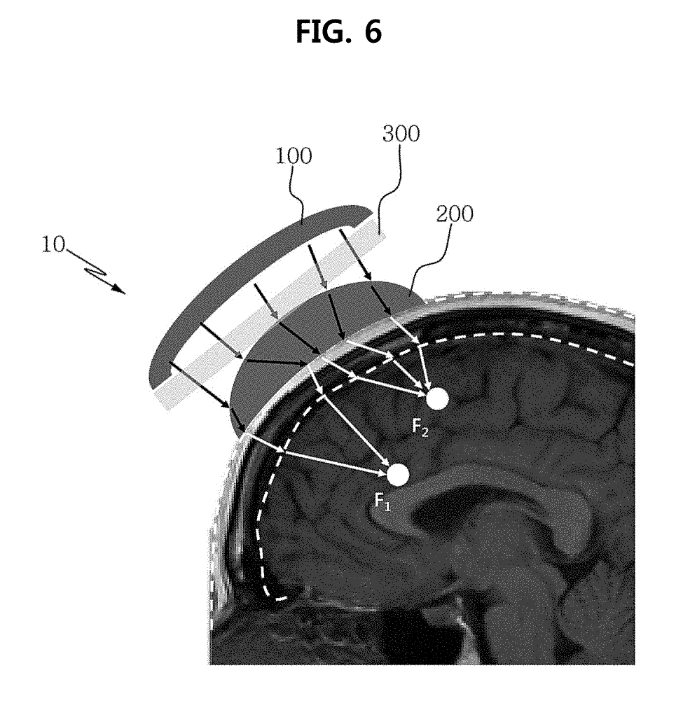

In an embodiment, the acoustic lens 200 may be customized to separately focus the outputted ultrasound onto at least two target focal points. Referring to FIG. 6, the ultrasound having passed through the acoustic lens 200 is focused onto a first target focal point F₁ and a second target focal point F2 along each path.

The conventional ultrasound stimulation apparatus needs to use multiple ultrasound sources (i.e., transducers) to simultaneously stimulate at least two target points, but with the ultrasound stimulation apparatus according to the embodiment of the disclosure, the ultrasound generated from one transducer 100 can be focused onto at least two target focal points F₁, F₂ as the ultrasound is allowed to pass through the pre-made customized acoustic lens 200.

To this end, the acoustic lens 200 may be customized along the target focal points set in consideration of the user's cranial shape pre-captured by CT or MRI as described above. According to the embodiment, the same effect as the use of multiple transducers or multi-array transducer can be obtained using one transducer, thereby reducing the cost.

As described above, the fixture 300 is configured to attach and detach the acoustic lens 200. Conventionally, even in the case of using an ultrasonic acoustic lens, it was general to additionally mount in front of a transducer to simply focus ultrasound, but according to the ultrasound stimulation apparatus of the disclosure, the acoustic lens is customized based on the cranial shape of the user (patient), and thus, in case that the apparatus is applied to a different part of the same user or a different user, replacing with different acoustic lenses customized for each case can significantly reduce the cost required for treatment, and can greatly improve the efficiency.

Referring to FIG. 7, in another embodiment, the fixture 300 may be formed with a structure allowing a second acoustic lens 250 to be inserted between the transducer 100 and the acoustic lens 200. For example, when the ultrasound stimulation apparatus is used to stimulate the brain of the user (patient), in some cases, it may stimulate different regions of the brain in a sequential order when necessary.

In this case, it may stimulate the first target focal point F₁ using only the first acoustic lens 200 first, and subsequently, may stimulate the second target focal point F2 by the second acoustic lens 250 additionally inserted into the fixture 300 to change the path of ultrasound. In this way, it is possible to stimulate many regions quickly by additionally inserting the pre-made customized second acoustic lens 250 without replacing the acoustic lens.

The focused ultrasound stimulation apparatus may further include a third acoustic lens which is attached in front of the transducer to improve the focality of ultrasound. In case that the apparatus includes the third acoustic lens, the first and the second acoustic lenses may be customized in further consideration of the refraction of ultrasound caused by the third acoustic lens, and in this case, the third acoustic lens is used to focus the ultrasound generated from the transducer more strongly.

In another embodiment, the third acoustic lens may be additionally inserted together with the second acoustic lens 250 to change the path of ultrasound so as to stimulate different target focal points. In this case, the third acoustic lens is used to focus the ultrasound onto more focal points (e.g., a third focal point F3), not simply improve the focality of ultrasound.

According to the embodiments, accuracy can be improved compared to the conventional ultrasound stimulation apparatus by focusing ultrasound onto a desired focus target using the acoustic lens customized based on the pre-captured user's cranial shape. Furthermore, by using acoustic lenses with different structures or inserting an additional acoustic lens, it is possible to stimulate multiple target focal points with a single transducer, so the same effect as the use of multiple transducers can be obtained. Additionally, because the fixture of the ultrasound stimulation apparatus has a structure that is easy to detach and attach or add the acoustic lens, extra costs incurred with the change in target patient or focus target can be reduced.

While the present disclosure has been hereinabove described with reference to the embodiments shown in the drawings, this is for illustration only and it will be appreciated by those having ordinary skill in the art that various modifications in details and embodiments may be made thereto. However, it should be noted that such modifications fall in the scope of technical protection of the present disclosure. Therefore, the true technical protection scope of the present disclosure shall be defined by the technical spirit of the appended claims.

What is claimed is:

1. A focused ultrasound stimulation apparatus, comprising:
a transducer which outputs ultrasound;
an acoustic lens which is placed in close contact with a user's skin, and is customized to focus the ultrasound onto a target focal point; and
a fixture for fixing the transducer and the acoustic lens to each other.

2. The focused ultrasound stimulation apparatus according to claim 1, wherein the acoustic lens is customized using a 3-dimensional (3D) printer based on a pre-captured user's cranial shape.

3. The focused ultrasound stimulation apparatus according to claim 1, wherein the acoustic lens is customized to focus the ultrasound separately onto at least two target focal points.

4. The focused ultrasound stimulation apparatus according to claim 1, wherein the fixture is configured to attach and detach the acoustic lens.

5. The focused ultrasound stimulation apparatus according to claim 1, wherein the fixture is configured to insert a second acoustic lens between the transducer and the acoustic lens,
wherein the second acoustic lens is customized to focus the ultrasound onto a target focal point different from the target focal point.

6. The focused ultrasound stimulation apparatus according to claim 1, wherein further comprises a third acoustic lens which is attached in front of the transducer to improve focality of the ultrasound.

7. The focused ultrasound stimulation apparatus according to claim 1, wherein the fixture is filled with a medium made of hydrogel or water.

8. The focused ultrasound stimulation apparatus according to claim 1, wherein the acoustic lens is made of at least one of polymers, elastomers and polydimethylsiloxane (PDMS).
